(19) United States
(12) Patent Application Publication
(10) Pub. No.: US 2026/0243781 A1
(43) Pub. Date: Aug. 20, 2026

(54) METHOD

(71) Applicant: Philip Morris Products S.A., Neuchatel (CH)

(72) Inventors: Laura ORTEGA TORRES, Neuchâtel (CH); Anna Grazyna GORALCZYK, Neuchâtel (CH); Jorge André MATIAS PEREIRA, Neuchâtel (CH); Neda HAGHAYEGH JAHROMI, Neuchâtel (CH)

(73) Assignee: Philip Morris Products S.A., Neuchatel (CH)

(21) Appl. No.: 19/475,326

(22) PCT Filed: Apr. 22, 2024
(86) PCT No.: PCT/EP2024/060965

§ 371 Date: Oct. 14, 2025

(30) Foreign Application Priority Data
Apr. 21, 2023 (EP) 23169335

(51) Int. Cl. G01N 33/68; G01N 21/64; G01N 33/84

(52) U.S. Cl. CPC G01N 33/6893; G01N 21/6486; G01N 33/84; G01N 2333/9116; G01N 2410/00; G01N 2800/7009

(57) ABSTRACT

A method of measuring oxidative stress in a cell or tissue sample, the method including the steps of: (i) treating the cell or tissue sample in a medium; (ii) collecting a portion of the medium; (iii) measuring amounts of glutathione in the cell or tissue sample; and (iv) measuring amounts of reactive oxygen species (ROS) in the media portion collected in step (ii), step (iii) being carried out immediately after steps (i) and (ii), and step (iv) being carried out simultaneously with step (iii) or within 6 hours of step (iii) including assay time if the collected media portion is stored at about 0-8° C. until step (iv) is carried out.

### FIELD OF THE INVENTION

The present invention relates to methods of measuring oxidative stress in cells or tissue. In particular, methods are described wherein amounts of reduced and oxidised glutathione and amounts of reactive oxygen species are measured in the same cell or tissue sample at the same time point.

### BACKGROUND TO THE INVENTION

Oxidative stress is a common hallmark of toxicity to environmental agents, while also being fundamental to intrinsic cellular processes. Oxidative stress can be described as a redox imbalance in a biological system arising from an excessive rate of production of oxidative species, specifically Reactive Oxygen Species (ROS), which fails to be counterbalanced by antioxidant defences, including reduced glutathione (GSH), which when oxidized becomes glutathione disulfate (GSSG). This disparity may cause damage to cells and cellular systems (Vona R, et. al., The Impact of Oxidative Stress in Human Pathology: Focus on Gastrointestinal Disorders. Antioxidants (Basel). 2021 Jan. 30; 10 (2): 201. doi: 10.3390/antiox10020201. PMID: 33573222; PMCID: PMC7910878) and oxidative stress has been shown to play an important role in the pathogenesis and progression of many human diseases (Forman H J, Zhang H. Targeting oxidative stress in disease: promise and limitations of antioxidant therapy. Nat Rev Drug Discov. 2021 September; 20 (9): 689-709. doi: 10.1038/s41573-021-00233-1. Epub 2021 Jun. 30. Erratum in: Nat Rev Drug Discov. 2021 August; 20 (8): 652. PMID: 34194012; PMCID: PMC8243062).

Most studies of oxidative stress provide an incomplete characterization of this cellular process, as per its scientifically accepted definition, by only evaluating either ROS or glutathione (GSH and/or GSSG) amounts, but not both biomarkers in the same biological sample at the same time point. The relevance of available oxidative stress-related data in the literature can often be debatable due to the lack of precision resulting from using different biological samples, variable time points, and low temporal resolution of response (For example Sies H, et. al., Defining roles of specific reactive oxygen species (ROS) in cell biology and physiology. Nat Rev Mol Cell Biol. 2022 July; 23 (7): 499-515 PMID: 35190722 and for a summary Frijhoff J, et. al., Clinical Relevance of Biomarkers of Oxidative Stress. Antioxid Redox Signal. 2015 Nov. 10; 23 (14): 1144-40. PMID: 26415143).

Most available methods of determining oxidative stress are not compatible with 3D tissue models, nor with high-throughput assessment activities, such as screening, due to low throughput capability and sensitivity to interference by testing compounds.

The present invention seeks to provide improved methods of measuring oxidative stress in which both ROS and glutathione (GSH and GSSG) amounts can be measured accurately, in the same tissue, and at the same timepoint.

### SUMMARY OF THE INVENTION

The present inventors have developed methods of measuring oxidative stress in a cell or tissue sample by optimising bioluminescence-based tests. Unexpectedly they have found it is possible to realise two different tests at the same time, in the same sample. That is, to perform two tests on the same endpoint for comprehensive and biologically relevant characterisation of oxidative stress.

Firstly, the inventors have improved assays utilising Luciferin-NT substrate and Glutathione S-Transferase (for example the GSH-Glo™ assay). Specifically, they have improved such assays, by including an additional step that is not part of the known protocol and uses a reducing reagent capable of the reduction of disulfide bonds (e.g., dithiothreitol (DTT), dithioerythritol, dithionite, sodium and potassium borohydride, tris(2-carboxyethyl) phosphine (TCEP), triphenylphosphine and tributylphosphine as described in, for example Monostori P, Wittmann G, Karg E, Turi S. Determination of glutathione and glutathione disulfide in biological samples: an in-depth review. J Chromatogr B Anal Technol Biomed Life Sci 2009; 877:3331-46.10.1016/j.jchromb.2009.06.016) for quantification of not only GSH (reduced form), but also total glutathione and GSSG (oxidized form), in the same sample. This optimization allowed for calculation of the GSH/GSSG ratio, which is an informative measure of the cellular redox status and a relevant oxidative stress biomarker. Although alternative kits for GSH/GSSG ratio assessment exist (for example Promega GSH/GSSG-Glo™), the improved method described herein provides a more relevant measurement of the ratio by assessing GSH (reduced form) and GSSG (oxidised form) from the same biological sample, with the additional benefit of a fifty per-cent cost reduction in terms of sample requirement.

Secondly, the inventors have further complemented a GSH/GSSG assay with an assessment of another oxidative stress biomarker, ROS. This can be used on the same sample as the glutathione assay, and thus enables a more holistic view of the oxidative stress of a cell or tissue sample to be obtained at a specific point in time. The term “biomarker” as used herein refers to a molecule, gene or characteristic by which a particular physiological process, especially a pathological process, can be identified.

The present invention allows measurements of multiple oxidative stress biomarkers to be taken over a time course (e.g., over a time period in which oxidative stress occurs to a cell or tissue sample, or to an individual from which cells are taken at time points). The timecourse is important in this context due to dynamic and transient nature of the biological phenomenon (Lushchak Ukr. Biochem. J. 2015; Volume 87, Issue 6, November-December, pp. 11-18).

In a first aspect there is described a method of measuring oxidative stress in a cell or tissue sample comprising the steps of: (i) treating the cell or tissue sample in a culture medium; (ii) collecting a portion of the medium; (iii) measuring amounts of glutathione (GSH, GSSG and total) in the cell or tissue sample; and (iv) measuring amounts of reactive oxygen species (ROS) in the media portion collected in step (ii); wherein step (iii) is carried out immediately after steps (i) and (ii); and wherein step (iv) is carried out simultaneously with step (iii) or within 6 hours of step (iii) including assay time if the collected media portion is stored at between 0 and 8° C., preferably between 2 and 6° C., most preferably at about 4° C. until step (iv) is caried out.

Suitably the ROS amount and/or glutathione amount is measured using luminescence.

Suitably the ROS amount and/or glutathione amount is measured quantitatively or qualitatively or relatively.

Suitably the medium of step (i) of the method comprises hydrogen peroxide (H₂O₂) substrate, most suitably ROS-Glo™ H₂O₂ substrate.

Suitably the treatment duration in step (i) of the method is 6 hours or less.

Suitably the amount of glutathione measured in step (iii) of the method is the sum of GSH (reduced form), and GSSG (oxidised form).

Suitably, the amount of glutathione measured in step (iii) takes into account the stoichiometry of glutathione redox cycle in which 2 GSH (reduced) molecules are oxidized into a single GSSG (oxidized) molecule, and conversely a single GSSG molecule is reduced back to two GSH molecules.

Suitably the GSH/GSSG ratio is calculated in step (iii) of the method.

Suitably the GSH/GSSG ratio is calculated using relative luminescence units (RLU) or glutathione molar concentration from glutathione standard curve.

Suitably a reducing agent, suitably a reduction agent capable of reduction of disulfide bonds is used to measure total glutathione amounts in step (iii) of the method.

In a further aspect, step (iii) of the method comprises at least the following steps:—
(a) place the cell or tissue sample in a well plate, add GSH-Glo™ reagent to each well and shake for about 2-10 minutes, preferably about 5 minutes at about 50-350 rpm, preferably about 300 rpm at room temperature to lyse the cells;
(b) add GSH-Glo™ reagent to standard curve wells in the assay plate;
(c) add GSH-Glo™ reagent to a reducing agent, and to one or more water vehicle background control wells in the assay plate;
(d) split each well containing the lysed cell or tissue sample into two wells in the assay plate;
(e) to measure total GSH, a reducing agent to one of each of the pairs of sample wells and to at least one reducing agent background control well;
(f) to measure reduced GSH, add water to the remaining sample wells which do not contain the reducing agent, and to the one or more water vehicle background control wells;
(g) shake all wells for about 1 minute at about 300 rpm at room temperature;
(h) Incubate all wells at room temperature;
(i) Add a reagent which can reveal the signal from luciferin-luciferase systems to the sample wells, to the at least one reducing agent well and one or more water vehicle background control wells and to the standard curve wells;
(j) shake all wells for about 1 minute at about 300 rpm at room temperature;
(k) incubate all wells at room temperature; and
(l) measure luminescence.

Suitably the reducing agent capable of reduction of disulfide bonds, suitably the reducing agent is dithiothreitol (DTT), dithioerythritol, dithionite, sodium borohydride, potassium borohydride, tris(2-carboxyethyl)phosphine (TCEP), triphenylphosphine or tributylphosphine for example TCEP Bond Breaker™ Tris (2-carboxyethyl)phosphine (TCEP).

Suitably step (l) is carried out using a luminescence plate reader.

In a further aspect, step (iii) of the method comprises at least the following steps:—
(A) Remove cell supernatant from the sample;
(B) Add passive lysis buffer to sample and shake for about 2-10 minutes preferably 5 min at about 50-350 rpm, preferably about 300 rpm;
(C) Divide the sample equally between two wells;
(D) In one well Luciferin-NT for quantification of total glutathione (GSH+GSSG);
(E) Add Luciferin-NT and of N-Ethylmaleimide (NEM) to the other well for quantification of oxidized GSSG;
(F) Incubate both wells for about 5 min shaking at about 300 rpm at room temperature;
(G) Add Luciferin Generation Reagent to each well. Shake for about 1 min;
(H) Incubate both wells at room temperature;
(I) Add a reagent which can reveal the signal from luciferin-luciferase systems to both wells and shake for about 1 min;
(J) Incubate both wells for about 14 min;
(K) Transfer sample wells to and assay plate containing a calibration curve; and
(L) Measure luminescence.

Suitably step (L) is carried out using a luminescence plate reader

Optionally the buffer in step (B) is kept cold. For example 0-6° C. Optionally the buffer in step (B) is kept on ice. Optionally the buffer used in step (B) is twice-concentrated (2×) to facilitate lysis of tissue samples.

In a further aspect, step (iv) of the method described herein comprises at least the following steps:—
(a) Add a control sample of medium only and an equal amount of compound interference controls to an assay plate;
(b) Add standard curve to at least 10 wells of the assay plate;
(c) Add a reagent which can reveal the signal from luciferin-luciferase systems to all wells, add D-cysteine if not already present in said reagent, preferably 0.01 to 20 mM, preferably 0.1 to 10 mM;
(d) Shake assay plate for about 1 minute at room temperature at about 300 rpm;
(e) Incubate the assay plate for about 20 minutes at room temperature.
(f) Measure luminescence.

Suitably wherein step (f) is carried out using a luminescence plate reader, suitably using the pre-defined “ROS-Glo™ H₂O₂ assay”.

An alternative Signal Enhancer solution can be optionally added on top to enhance signal in step (c) such as Pierce™ Firefly Signal Enhancer (Thermofisher) (https://www.thermofisher.com/order/catalog/product/16180).

A further aspect of the invention comprises a kit comprising materials to complete one or more methods of the invention, and optionally instructions. Suitably said materials comprise the reagents required for one or more methods of the invention.

In a further aspect the kit of the invention comprises GSH-Glo™ assay from catalogue #V6911 or V6912 as described in technical manual #TB369 from Promega, additionally or alternatively said kit comprises the GSH-Glo™ assay from catalogue V6912 as described in technical manual #TB344 from Promega, plus a reducing agent, and optionally instructions. Suitably said reducing agent is capable of reduction of disulfide bonds, suitably the reducing agent is dithiothreitol (DTT), dithioerythritol, dithionite, sodium borohydride, potassium borohydride, tris(2-carboxyethyl)phosphine (TCEP), triphenylphosphine or tributylphosphine, suitably the reducing agent is TCEP.

Suitably the kit of the invention further comprises materials to complete the ROS-Glo™ H₂O₂ assay and optionally further instructions. Suitably the kit comprises the TM391 ROS-Glo™ H₂O₂ assay kit from catalogue #G8820 and G8821 as described in technical manual TM391 from Promega.

### Some Advantages

The present invention has the advantage of enabling a comprehensive assessment of oxidative stress in a tissue or cell sample by assessing two major oxidative stress biomarkers, ROS amounts and GSH/GSSG ratio, in the same sample at the same time point.

The present invention allows multiple measurements of said oxidative stress biomarkers to be taken over a time course or period. For example, measurements could be taken at many points over a time period in which oxidative stress occurs to cells or tissue samples, or at many times to an individual from which cell samples are taken. For multiple time points a single sample is needed for the ROS assay if the non-lytic variant is used, but multiple samples are needed for the glutathione assays (one for each time point).

This approach can be applied to both 2D and 3D cell culture models for time-course experiments, and in a high-throughput manner.

The methods of the present invention are less susceptible to compound interference in the context of chemical assessment when compared to other methodologies, as they are based on luminescent readouts instead of the majority of fluorescent or colorimetric available readouts.

### BRIEF DESCRIPTION OF THE DRAWINGS

FIG. 1 is a diagram comparing known methods of assessing oxidative stress with the methods of the invention.

FIG. 2 is a schematic showing the conversion of a luciferin derivative into luciferin in the presence of glutathione, catalyzed by glutathione S-transferase.

FIG. 3 shows in diagrammatic form the process of the known GSH-Glo™ assay (top, section A) compared to the methods of the invention (bottom, section B).

FIG. 4A is a graph showing the amount of GSH (reduced form) measured in a sample using the GSH-Glo™ kit.

FIG. 4B is a graph showing the amount of GSH, GSSG and combined GSH+GSSG measured in a sample using the modified GSH-Glo™ kit in the method of the invention.

FIG. 4C is a graph showing the GSH/GSSG ratio obtained from the results showing in FIG. 4B.

FIG. 5 is a diagram showing the ROS-Glo™ non-lytic protocol.

FIG. 6 shows the chemistry involved in the ROS-Glo™H₂O₂ assay.

FIG. 7 is a schematic showing the experimental protocol which obtained the results of FIG. 8.

FIG. 8 top row shows the results of carrying out the modified GSH/GSSG-Glo™ kit and ROS-Glo™ H₂O₂ assay in the method of the invention separately, and at different time points on the same sampleOn the top row we see a comparison carrying out the standard (unmodified) assays on the same cell cultures over the same time points.

FIG. 9 shows dose dependent effects on glutathione levels, ROS and cell viability when carrying out the modified GSH/GSSG-Glo™ kit and ROS-Glo™ H₂O₂ assay in the method of the invention on the same samples as shown in FIG. 8.

FIG. 10A is a diagram showing the characteristics of fully-differentiated 3D bronchial cultures from primary human cells, including cell type composition and culturing conditions in vitro. This is modified from Modified from Kirkpatrick and Millard, BioRxiv. 2020 https://doi.org/10.1101/2020.09.16.300483.

FIG. 10B is an enlargement of a section of FIG. 10A.

FIG. 11 is a further schematic showing how GSH/GSSG and ROS assays can be adapted to 3D bronchial cultures

FIG. 12 is a collection of graphs illustrating experimental data obtained by using the methods of the invention on 3-D bronchial cultures.

FIG. 13 is a collection of graphs showing experimental data using the method of the invention on a A549 cell line.

FIG. 14 is a schematic showing the experimental protocol which obtained the results of FIG. 15

FIG. 15 shows further experimental data obtained by using the methods of the invention in 3d bronchial cultures.

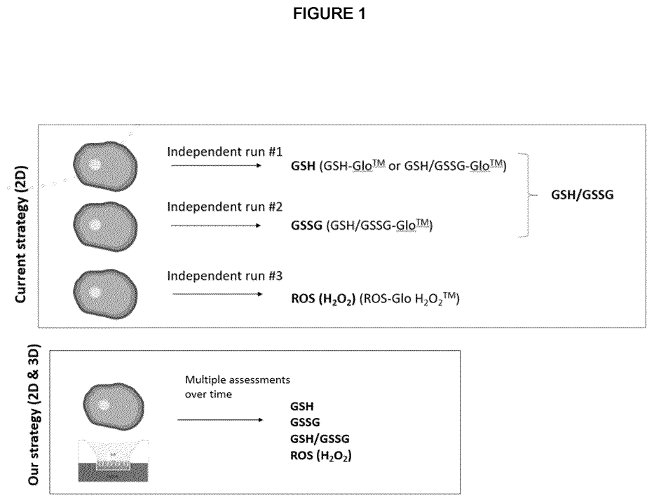

### DETAILED DESCRIPTION

The practice of the present disclosure employs, unless otherwise indicated, conventional techniques of engineering, micro-engineering, microbiology, cell biology and biochemistry. Such techniques are explained fully in the literature, such as, in Molecular Cloning: A Laboratory Manual, second edition (Sambrook et al., 1989) Cold Spring Harbor Press; Oligonucleotide Synthesis (MJ. Gait, ed., 1984); Methods in Molecular Biology, Humana Press; Cell Biology: A Laboratory Notebook (J. E. CelMs, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, I B. Griffiths, and D. G. Newell, eds., 1993-8) J. Wiley and Sons; Methods in Enzymology (Academic Press, Inc.); Current Protocols in Molecular Biology (F.M. Ausubel et al., eds., 1987); PCR: The Polymerase Chain Reaction, (Mullis et al., eds., 1994). Procedures employing commercially available kits and reagents will typically be used according to manufacturer-defined protocols unless otherwise indicated.

The technical terms and expressions used herein are generally to be given the meaning commonly applied to them in the pertinent art of molecular biology, microbiology, cell biology and biochemistry. All of the following term definitions apply to the complete content of this application.

As used herein, the singular forms “a”, “an”, and “the” include both singular and plural referents unless the context clearly dictates otherwise.

The term “and/or” means (a) or (b) or both (a) and (b).

The terms “comprising”, “comprises” and “comprised of” as used herein are synonymous with “including”, “includes” or “containing”, “contains”, and are inclusive or open-ended and do not exclude additional, non-recited members, elements or method steps.

The term “consisting of” means that additional components are excluded and has the recited elements only and no more.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosure. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

The term “at least” include not less than the minimum stated, but can encompass more/additional steps or items.

The first aspect of the invention is method of measuring oxidative stress in a cell or tissue sample comprising the steps of: (i) treating the cell or tissue sample in a medium; (ii) collecting a portion of the medium; (iii) measuring amounts of glutathione in the cell or tissue sample; and (iv) measuring amounts of reactive oxygen species (ROS) in the media portion collected in step (ii); wherein step (iii) is carried out immediately after steps (i) and (ii); and wherein step (iv) is carried out simultaneously with step (iii) or within 6 hours of step (iii) including assay time if the collected media portion is stored at about 1-6° C., suitably about 4° C., until step (iv) is caried out. The media portion collected in step (ii) contains medium and cells.

The term “oxidative stress as used herein refers to a disturbance in the balance between the production of reactive oxygen species (referred to herein as “ROS”) and antioxidant defences in a cell which detoxify reactive species or repair damaged caused by them. In other words, it occurs when the normal redox state of cells is disturbed, and this can cause toxic effects through the production of free radicals that can damage the cell components such as DNA, and disrupt cellular functions such as respiration (See for example Lushchak Ukr. Biochem. J. 2015; Volume 87, Issue 6, November-December, pp. 11-18). Oxidative stress may also cause lipid peroxidation, protein carboxylation and apoptosis (cell death). “Free radicals” are any molecular species capable of independent existence that contains an unpaired electron in an atomic orbital. ROS are a subset of free radicals that contain oxygen. Examples of ROS include superoxide radical (O₂⁻), hydroxyl radical (OH) and hydrogen peroxide (H₂O₂). Oxidative stress can be caused by environmental hazards such as excess meals, but is also part of many cell processes such as oxidative phosphorylation. Oxidative stress is thought to be associated with the development of many diseases in humans, especially neurodegenerative diseases such as Parkinson's disease, Alzheimer's disease and multiple sclerosis. However, ROS can have beneficial uses in the immune system. There are many ways of measuring oxidative stress, some of which are summarised in Table 1 below.

TABLE 1

| Biomarkers | | In vitro method |
|---|---|---|
| Reactive | Free radicals | Free radical species by electron spin |
| Oxygen | | resonance; radical spin trapping |
| Species | Intracellular ROS | Intracellular ROS with fluorescent probes |
| | | (e.g., dihydroethidium [DHE], |
| | | dichlorofluorescin diacetate [DCFDA] or |
| | | dihydrorhodamine [DHR]) or luminescent |
| | | probes (ROS-Glo H₂O₂ ™) |
| Antioxidant | Reduced glutathione | Detection of reduced GSH by colorimetric, |
| capacity | (GSH) | fluorescence, luminescence-based assays |
| | | (GSH-Glo ™) |
| | Glutathione redox ratio | Detection of GSH and GSSG by colorimetric, |
| | (GSH/GSSG) | fluorescence, luminescence-based assays |
| | | (GSH/GSSG-Glo ™ and GSH-Glo ™) |
| | Total antioxidant capacity | Measurement of both small molecules and |
| | | proteins antioxidants using colorimetric |
| | | assays |
| | Antioxidant enzyme activity | e.g., SOD or Catalase activity assays |
| Cellular | Lipid peroxidation | Detection of malondialdehyde by ELISA and |
| damage | | MS |
| | Protein oxidation | Detection of protein carbonylation (3- |
| | | nitrotyrosine) by ELISA and MS |

“GSH” as referred to herein is the reduced form of glutathione made from three amino acids: cysteine, glutamate, and glycine, having the structure shown below.
!

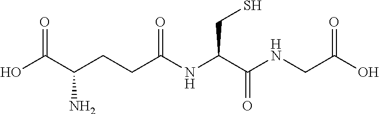 [SMILES: N[C@@H](CCC(=O)N[C@@H](CS)C(=O)NCC(=O)O)C(=O)O]

All animal cells are capable of producing GSH, but in mammals GSH is mainly produced by the liver. GSH is involved in many bodily processes, such as tissue building and repair, making chemicals and proteins needed in the body, and in immune system function.

“Glutathione disulfide” or “GSSG” as referred to herein, is the oxidised state of GSH as a disulfide derived from two glutathione molecules, having the structure shown below.

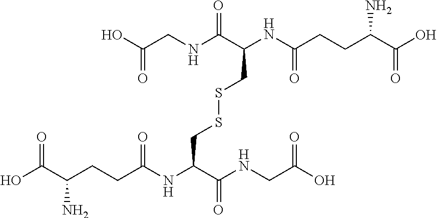 [SMILES: N[C@@H](CCC(=O)N[C@@H](CSSC[C@H](NC(=O)CC[C@H](N)C(=O)O)C(=O)NCC(=O)O)C(=O)NCC(=O)O)C(=O)O]

(image by Fvasconcellos 23:40, 1 Oct. 2007 (UTC)-Own work, Public Domain, https://commons.wikimedia.org/w/index.php?curid=2843592)

In living cells, glutathione disulfide is reduced into two molecules of glutathione with reducing equivalents from the coenzyme NADPH.

GSH protects cells by reducing ROS This conversion is illustrated by the reduction of peroxides as per the equation below:

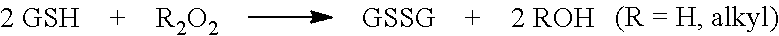

This reaction can be catalysed by enzymes such as glutathione peroxidases and peroxiredoxins.

The term “glutathione” can refer to GSH (reduced form) or GSSG (oxidised form), but unless specified refers herein to the total sum of reduced GSH and oxidised GSSG.

The GSH:GSSG ratio is an important bioindicator of cellular health, with a decreasing ratio being indicative of oxidative stress.

The terms “cell culture” and “tissue culture” are used interchangeably herein. Cell culture generally refers to the removal of cells from a tissue (i.e. a cell or tissue sample) prior to growth in an artificial environment. The cells to be cultured can be removed directly from a tissue containing the cell to be cultured and optionally treated with enzymatic or mechanical means prior to culture. As an alternative, the cells to be cultured can be derived from a prior established strain or cell line. A section of cells or tissue from a culture to be tested is known herein as a “cell sample” or “tissue sample” or “biological sample”, and these terms are used herein interchangeably. A “cell line” is a collection of cells originating from one cell that can proliferate indefinitely in a medium.

The present disclosure utilises various sources of cells. In one embodiment, the present disclosure excludes the step of isolating or obtaining a cell sample from a subject. The cells can be cryopreserved. The cells can be in 3-dimensional cell culture. The cells can be in the form of tissues. The cells can be in the form of spheroids. The cells can be actively dividing. The cells can be cultured in the cell culture device in the presence of cell culture medium (for example, comprising nutrients (for example, proteins, peptides, amino acids), energy (for example, carbohydrates), essential metals and minerals (for example, calcium, magnesium, iron, phosphates, sulphates), buffering agents (for example, phosphates, acetates), indicators for pH change (for example, phenol red, bromo-cresol purple), selective agents (for example, chemicals, antimicrobial agents), etc.). A single cell culture medium can be used to grow cells of the same or different types. Different cell culture media can be used to grow different types of cells. Since the cell culture media are circulated in accordance with the present disclosure then mixing of the different cell culture media will occur. In some experiments herein “NHBE cells” have been used. “NHBE” stands for “Normal human bronchial epithelial” cells. These are primary cells isolated from the epithelial lining of the airway above the bifurcation of the lungs. Said NHBE cells are commonly used in respiratory disease research. In some experiments herein, an “A549” cell line (also referred to as “A549 cells”) has been used. This is a cell line of human adenocarcinome alveolar basal epithelial cells of lung tissue origin. This cell line is commonly used in the development of drug therapies, especially cancer. In some embodiments, one or more agents are included in the cell culture medium or cell culture media. Cells can be isolated from a tissue or a fluid using methods that are well known in the art. Cells can be differentiated from stem cells-such as embryonic stem cells or induced pluripotent stem cells, or directly differentiated from somatic cells. The cells may be natural cells or altered cells (for example, a cell comprising one or more non-natural genetic alteration). The cell may be a disease cell or disease model cell. For example, the cell can be a cancer cell or a cell that can be induced to a hyper-proliferative state (e.g., transformed cells). The terms “medium”, “media”, and “culture medium/media” can be used interchangeably herein. In some steps of the methods described herein the medium used comprises hydrogen peroxide (H₂O₂) substrate. The term “substrate” as used herein refers to an addition to the medium which can act as a probe, for example by inducing a colour change or luminescence. Most suitably the medium used herein comprises a substrate which reacts directly with H₂O₂ to create luminescence, most suitably ROS-Glo™ H₂O₂ substrate. ROS-Glo™ H₂O₂ substrate reacts directly with H₂O₂ to create a Luciferin Precursor.

Cells may be or may be derived from human or animal subjects or from human or animal cells, including any of a number of mammalian species, suitably human, but including rat, mouse, pig, rabbit, and non-human primates and the like. Cells and cell lines can be obtained from commercial sources. Cells may be from or derived from any desired tissue or organ type, including but not limited to, adrenal glands, bladder, blood vessel, bone, bone marrow, brain, cartilage, cervix, cornea, endometrium, oesophagus, gastrointestinal system, immune system (e.g., T lymphocytes, B lymphocytes, leukocytes, macrophages, and dendritic cells), liver, lung, lymphatic system, muscle (e.g., cardiac muscle), nervous system, ovaries, pancreas (e.g., islet cells), pituitary gland, prostate, kidney, salivary gland, skin, tendon, testis, and thyroid.

Lung cells—including lung epithelial cells—are one cell type of particular interest. Bronchial and/or other airway epithelial cells are of particular use in the present disclosure. Human bronchial epithelial cells can be collected by brushing donor lungs during a bronchoscopy procedure. In one embodiment, the lung cells are Normal Human Bronchial Epithelial (NHBE) cells. The lung epithelial cells can be cultured as a monolayer of undifferentiated cells or further developed into an organotypic lung epithelium-like tissue at an air-liquid interface. Lung epithelial cells can be obtained from human or animal subjects with different pathologies, including subjects that are classified as smokers or non-smokers.

In one embodiment, the cells used are hepatocytes. Hepatocytes are cells of the liver, which make up 70-85% of the liver's cytoplasmic mass. The functionality of hepatocytes is highly dependent on their capacity to form a polar phenotype, which is only established in 3-dimensional culture. One source of liver cells is primary hepatocytes which are an in vitro model widely used to investigate numerous aspects of liver physiology and pathology. The technique used to isolate human hepatocytes can be based on a two-step collagenase perfusion of a donated liver. However, these cells do not express metabolic enzymes for more than 5 days. Another limitation is their short viability. These drawbacks can be overcome by the use of alternative, long-lived liver cell lines-such as human or animal hepatic progenitor cell lines. One such example of a human hepatic progenitor cell line is the HepaRG™ cell line (ThermoFisher Scientific). HepaRG™ cells retain many characteristics of primary human hepatocytes. They have greater liver-specific and metabolic gene expression compared to primary hepatocytes and a longer lifespan. Reorganisation of HepaRG™ cells in 3-dimensional spheroids further increases both the lifespan and metabolic capabilities, suggesting that spheroids may provide a better alternative in vitro liver model for toxicity testing. Liver spheroids can also be created with a mixture of primary hepatocytes and liver stellate cells or primary hepatocytes and adipose tissue-derived stem cells.

In one embodiment, the lung cell is a lung epithelial cell—such as a bronchial and/or other airway epithelial cell.

In one embodiment, the liver cell is a hepatocyte, suitably, a HepaRG cell.

The use of combinations of any of the cells described herein is contemplated. The use of combinations of any of the cells described herein in the methods of the invention. One exemplary combination of cells is the combination of liver and lung cells. The combination of a lung epithelial cell—such as a bronchial and/or other airway epithelial cell, and a liver cell—such as a HepaRG™ cell, is contemplated. Additional cells can be used together with this combination if required.

The different cells of the combination can be cultured in separate wells.

The present disclosure incorporates the use of “3-dimensional cell culture” (or “3D cell culture” used herein interchangeably), which includes any method that provides for the culture of a cell in 3 dimensions, with or without the use of a matrix or scaffold—such as the permeable membrane in the insert. A number of different 3-dimensional cell culture methods have been developed, including spheroid cultures and organotypic cultures. 3-dimensional cells can be grown and/or maintained in the cell culture device described herein. The tissue or cell sample used in any method described herein may be cultured in a 3-dimensional cell culture before, during or after the methods of the invention are carried out.

The term “spheroid” assumes the meaning as normally understood in the art which is either a single cell that divides into a ball of cells in 3-dimensions, or an aggregation of multiple cells in 3-dimensions, either with or without the use of a matrix or scaffold to support 3-dimensional cell growth within the spheroid. The 3-dimensional spheroid can be an adherent spheroid or a spheroid grown in suspension.

In some embodiments, a spheroid contains a single cell type. In some embodiments, a spheroid contains more than one cell type. In some embodiments, where more than one spheroid is grown, each spheroid is of the same type, while in other embodiments, two or more different types of spheroids are grown.

3-dimensional spheroids more closely resemble in vivo tissue in terms of their cellular communication and development of extracellular matrix. This matrix assists the cells in moving within the spheroid similar to the way cells would move in living tissue. The spheroids are thus much improved models for differentiation, survival, cell migration, cell polarisation, gene expression and growth.

Spheroids can be harvested and studied using various methods well known in the art, including colorimetric, fluorescence, and luminescence assays measured with a plate reader or they can be readily observed by microscopy. Additional techniques include Western, Northern or Southern blot, histological techniques (for example, immunohistrochemistry, in situ hybridization, immunofluorescence) and the like. The use of optical imaging methods—such as inverse bright field microscopy and fluorescence microscopy, is also contemplated.

Applications of the use of 3-dimensional spheroids include the study of the proliferation of cells and tissues in vitro in an environment that more closely approximates that found in vivo, the screening of compounds, toxicology assays, cell therapy, cell delivery, agent delivery, biochemical replacement, production of biologically active molecules, tissue engineering, biomaterial, and clinical trials and the like.

The use of spheroids in 3-dimensional cell culture is generally reviewed in Expert Opin. Drug Discov. (2015) 10, 519-540.

3-dimensional organ culture systems, especially those in miniaturised form, can be used in the present disclosure as they allow the study of how organs function on a micro-scale. Response to certain stimuli, response to one or more agents, and pharmacokinetic behaviour of such agents can be studied. Miniaturised 3-dimensional cell culture systems allow the combined study of groups of cells or organs. This allows the complexity of interaction between different tissues to be reproduced. The 3-dimensional organ culture can be organotypic, which means that it seeks to reproduce major functions of an organ or organ system. A miniaturised fluidic system interconnecting the wells is also contemplated.

In one aspect, there is provided a culture of spheroids in which the spheroids are in the form of individualised single spheroids after 5 hours of culture or after 5 days of culture. In other words the spheroids are not agglomerated or fused. Said culture may be used as the cell or tissue sample in any of the methods described herein.

Liver—The liver plays a central role in detoxification, metabolism of carbohydrates, lipids and proteins as well as biotransformation of endogenous and exogenous substances. Liver functionality is closely linked to the assembly of highly specialised cells, the majority of which are hepatocytes, embedded in a complex 3-dimensional structure made up of so-called lobules. Biotransformation of compounds usually results in non-toxic and more soluble metabolites, however, occasionally, more toxic metabolites may be formed causing hepatotoxicity.

Hepatocytes can be maintained in 3-dimensions via various methods, including the use of sandwich culture, solid scaffold materials—such as polystyrene scaffolds, hydrogels-such as collagen type-I, or self-assemble into spheroids.

Whilst the use of freshly isolated primary human hepatocytes may be the preferred liver cell type, their availability is limited. Other choices of human liver cell lines include HepG2 and Hep2/C3A cells. A particularly suitable cell source is the HepaRG™ cell line. Other sources of human hepatocytes are human embryonic stem cell (hESC)-derived hepatocytes and hepatocytes derived from induced pluripotent stem cells (iPSC).

In one embodiment, the spheroid is or is derived from a liver cell to form a 3-dimensional liver spheroid. Such liver spheroids can be prepared using various methods that are known in the art and described in, for example, ALTEX (2014) 31, 441-477 and Toxicol. Sci. (2013) 133, 67-78.

Lung—As the morphology of the respiratory tract changes from the upper to the lower airways, many different cell culture models have been established using primary airway epithelial cells or cell lines and are contemplated for use in the present disclosure. The choice of exactly which cell or cell line to use will depend on the area of interest of the respiratory tract for a given study.

Since the lung surface is exposed to air, the cell model can be cultured at the air-liquid interface to mimic the lung more realistically.

In one embodiment, the lung 3-dimensional culture is or is derived from a lung cell to form a 3-dimensional organotypic tissue. Such lung tissues can be prepared using various methods that are known in the art, such as those described in ALTEX (2014) 31, 441-477 and Toxicol. (2013) 133, 67-78.

The cell cultures used in the disclosed methods can also be cultured on plates in conventional 2-dimensional cell culture. For example in a petri dish.

The term “treating” as used herein refers to adding any chemical, nutrient or composition to the cell sample or culture, alternatively or in addition to changing any environmental conditions such as temperature. The treatment may be added via bathing or pouring for example. Treatments may be added to a medium or a treatment may comprise a specific medium. For example, in some embodiments the methods described herein treat tissues a medium which reacts directly with H₂O₂ to create a Luciferin Precursor

Treatment may be basolateral (applied to the base, base membrane or lateral side of cells or tissue) or apical (applied to the top, outer or air-facing surface of cells or tissue. Basolateral treatment may allow better control of chemical compound concentration. However, in some embodiments apical treatment is more physiologically relevant, for example for air-liquid interface 3D tissues, or submerged tissues.

The term “collecting” as used herein refers to removing all or some of a sample, liquid or solid. For example, taking some cells from a culture for use in the next step of a method.

The term “portion” as used herein refers to any part of a whole. 1%, 2%, 5%, 10%, 20%, 25%, 30%, 50%, 75%, 80% or any amount can constitute a portion.

The term “measuring” as used herein refers to any way of ascertaining the size, amount or degree of something. Measuring (to produce measurements) can be carried our quantitatively or qualitatively or relatively. Measurements can be quantitative, qualitative or relative.

Specifically in the methods described herein it refers to measuring the ROS amount and/or the glutathione (GSH and GSSG or both) amount. These measurements are indicative of oxidative stress as discussed above.

In some embodiments of the methods described herein, the ROS amount and/or the glutathione amount is measured quantitatively or qualitatively or relatively. In some embodiments ROS amount and/or GSH amount is measured using luminescence. Methods can measure GSH (reduced), GSSG (oxidised) or total glutathione.

Measurements, suitably luminescence measurements described herein, can be relative to a standard curve or background,

In some embodiments luminescence is measured quantitatively or qualitatively or relatively.

The term “amount” as used herein refers to a quantity of something, especially the total of a thing or things in number, size, value, or extent.

The phrase “immediately after” as used herein refers to consecutively within a short amount of time, suitably within 10 minutes, suitably within 30 minutes, suitably within 60 minutes.

The term “simultaneously” as used herein means at the same time or within 5, 10, 20, 30 minutes or less.

Suitably the treatment duration in step (i) of the method described herein is 6 hours or less, preferably 5 hours or less, preferably 4 hours or less, preferably 3 hours or less, preferably 2 hours or less, most preferably 1 hour or less.

The term “luminescence” as used herein refers to the emission of light by a substance as a result of a chemical or enzymatic reaction (bioluminescence). Luminescence detection is optically simpler than fluorescence detection as it does not require a light source or specific optics for excitation.

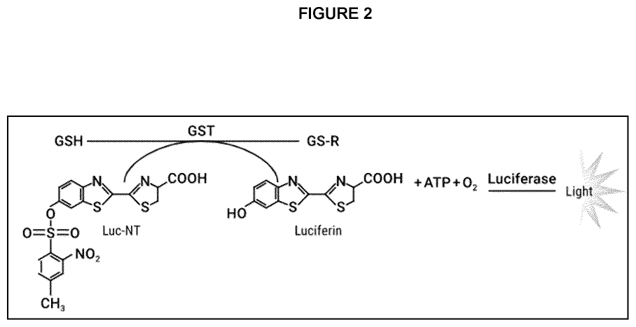

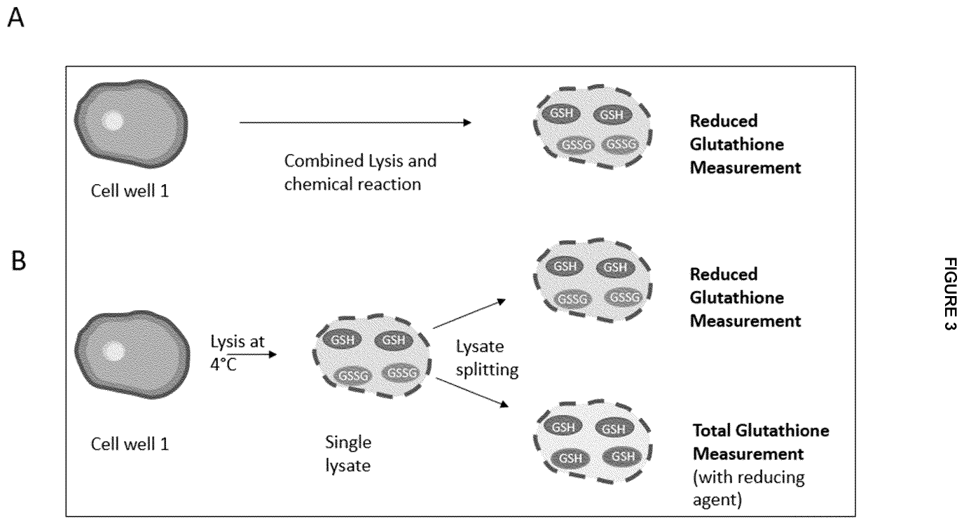

The Glutathione Assays of the invention are luminescence-based assays for detecting and quantifying GSH and/or GSSG. They are based on the conversion of a luciferin derivative into luciferin in the presence of glutathione, catalyzed by glutathione S-transferase (GST). The signal generated in a coupled reaction with firefly luciferase is proportional to the amount of glutathione present in the sample. FIG. 2 illustrates the reaction involved.

Suitably the amount of glutathione measured in step (iii) of the method is the sum total of reduced GSH, and oxidised GSH (GSSG).

Suitably the GSH/GSSG ratio is calculated in step (iii) of the method.

Suitably the GSH/GSSG ratio is calculated using relative luminescence units (RLU). RLU are the units used for most luminescence measurements. RLU do not have any physical meaning and are often not comparable between different instruments.

Suitably a reducing agent is used to measure GSH amounts in step (iii) of the method. Suitably said reducing agent is capable of reduction of disulfide bonds, suitably the reducing agent is dithiothreitol (DTT), dithioerythritol, dithionite, sodium borohydride, potassium borohydride, tris(2-carboxyethyl)phosphine (TCEP), triphenylphosphine or tributylphosphine, Suitably the reducing agent is TCEP, suitably Bond Breaker™ TCEP.

The term “room temperature” as used herein is about 18-25° C., suitably about 18-22° C. suitably about 20-22° C.

The term “incubate” as used herein refers to a period of time wherein samples and/or regents are left to progress and/or react and/or develop. Incubation may take place in the dark or otherwise reduced light.

In a further aspect, step (iii) of the method of the invention comprises at least the following steps:—
(a) place the cell or tissue sample in a well plate, add GSH-Glo™ reagent to each well and shake for about 5 minutes at about 300 rpm at room temperature to lyse the cells;
(b) add GSH-Glo™ reagent to standard curve wells in the assay plate;
(c) add GSH-Glo™ reagent to a reducing agent and one or more water vehicle background control wells in the assay plate;
(d) split each well containing the lysed cell or tissue sample into two wells in the assay plate;
(e) to measure total glutathione, add the reducing agent to one of each of the pairs of sample wells and to at least one reducing agent background control well;
(f) to measure reduced GSH, add water to the remaining sample wells which do not contain the reducing agent, and to the one or more water vehicle background control wells;
(g) shake all wells for about 1 minute at about 300 rpm at room temperature;
(h) Incubate all wells at room temperature;
(i) Add a reagent which can reveal the signal from luciferin-luciferase systems to the sample wells, to the at least one reducing agent and one or more water vehicle background control wells and to the standard curve wells;
(j) shake all wells for about 1 minute at about 300 rpm at room temperature;
(k) incubate all wells at room temperature; and
(l) measure luminescence.

Suitably the shaking in step (a) can last from about 1 to about 8 minutes, at about 250-350 rpm, most suitably 390-310 rpm.

Suitably the shaking in step (j) can last from about 10 seconds to about 5 minutes, preferably 1-2 minutes, at about 50-350 rpm, most suitably 390-310 rpm.

Suitably a reducing agent is used step (c), (e), (f) and (i) of the method. Suitably said reducing agent is capable of reduction of disulfide bonds, suitably the reducing agent is dithiothreitol (DTT), dithioerythritol, dithionite, sodium borohydride, potassium borohydride, tris(2-carboxyethyl)phosphine (TCEP), triphenylphosphine or tributylphosphine. Suitably said reducing agent is TCEP, suitably Bond Breaker™ TCEP.

Suitably step (l) is carried out using a luminescence plater reader, suitably using the pre-defined protocol.

Suitably “GSH-Glo™ reagent” is a Glutathione S-transferase (GST reaction buffer used in the Promega GSH-Glo™ assay and kit thereof (catalog #s V6911, V6912, V6611 and V6612). Alternatively, this can be substituted by recombinant GST and it's reaction buffer from any other supplier, A Luciferen-NT substrate needs to be added to this reaction buffer. An alternative to Luciferen-NT, found in the same Promega kit, can be produced by the caged strategy of luciferin with a nitrobenzenesulfoamide group.

“Luciferin detection agent” as used herein refers to the Promega product catalog #V8921 or V8920. It can be substituted by equivalent reagents from other suppliers including Luciferase, esterase, ATP and Mg2+ which were developed to reveal signal from Luciferin-Luciferase (Ultra-Glow Luciferase) systems.

Luciferin detection agent can also be substituted by firefly luciferase (0.0001 to 10 units/mL, preferably 0.1 to 1 unit/mL) diluted in a buffer solution such as 1× Tris-glycine buffer, pH 7.6 or 0.1 M Tris-HCl, pH 8, with the addition of the following components:
EDTA 0.1 mM-2 mM
Mg²⁺ in the form of MgSO4 (8-16 mM)
ATP: 0.1 to 10 mM, preferably 1 to 5 mM
Esterase: 10-6 to 10-2 units/mL, preferably 10-4 to 10-3 units/mL

Individual components can be acquired separately or already in the form of kits.

In a further aspect, step (iii) of the method comprises at least the following steps:—
(A) Remove cell supernatant from the sample;
(B) Add passive lysis buffer to sample and shake for about 2-10 minutes, preferably about 5 min at about 50-350 rpm, preferably 300 rpm;
(C) Divide the sample equally between two wells;
(D) In one well add Luciferin-NT for quantification of total GSH;
(E) Add Luciferin-NT and of N-Ethylmaleimide (NEM) to the other well for quantification of oxidized GSSG;
(F) Incubate both wells for about 5 min shaking at about 300 rpm at room temperature;
(G) Add Luciferin Generation Reagent to each well. Shake for about 1 minute;
(H) Incubate both wells at room temperature;
(I) Add a reagent which can reveal the signal from luciferin-luciferase systems to both wells and shake for about 1 minute;
(J) Incubate both wells for about 14 minute;
(K) Transfer sample wells to and assay plate containing a calibration curve; and
(L) Measure luminescence.

Suitably the shaking in steps (B) and (F) can last from about 2 to about 10 minutes, preferably at about 50-350 rpm, most suitably 300-310 rpm.

Suitably the method steps B to E are carried out in cold conditions such as on ice, suitably at about 1-10° C., suitably at about 1-5° C., most suitably at about 4° C.

Suitably the shaking in steps G and I is carried out at 50-350 rpm, most suitable 300 rpm.

Suitably step (L) is carried out using a luminescence plate reader.

A “passive Lysis buffer”, as used herein, is specifically formulated to promote rapid lysis of cultured cells, suitably mammalian cells, without the need for scraping adherent cells or performing freeze-thaw cycles. For example, Passive Lysis Buffer, 5× from Promega, Cat #E1941, which elicits only minima; coelenterazine auto-luminescence, making it particularly suitable for assays such as those described herein.

“Luciferin Generation Reagent” as used herein is a buffer which, other availability through the Promega kits disuse therein, can be prepared with purified Glutathione-S-Transferase, 2.5 mM Dithiothreitol (DTT) available from many suppliers. This is diluted in Dulbecco's Phosphate Buffered Saline or 100 mM NaH₂PO₄, PH 7.0 and 1 mM EDTA.

A “calibration curve” as used herein is a plot of how an instrument responds to an analytical signal. It is a method for determining the concentration of a substance in an unknown sample by comparing the unknown to a set of standard samples of known concentration. It can also be known as a standard curve. In the assays herein disclosed, multiple wells in any sample plate may be assayed in order to determine a calibration curve to compare the results (usually luminescence) of the cell or tissue samples against. For example, for the methods described herein, prepare a standard curve using a GSH standard solution (Glutathione, 5 mM) to facilitate conversion of luminescence (in RLU) to GSH and/or GSSG concentration. Dilute the Glutathione, 5 mM stock (1:100) in water, and then perform serial 1:1 dilutions by combining an equal volume of Glutathione and an equal volume of water. Transfer 10 μL of each diluted standard to the appropriate wells for the assay. The final concentration of Glutathione, 5 mM, will range from 0 μM to 5 μM.

Generic principles for calibration curve preparation are described in:
FDA Bioanalytical Method Validation Guidance for Industry https://www.fda.gov/files/drugs/published/Bioanalytical-Method-Validation-Guidance-for-Industry.pdf
and/or
ICH guideline M10 on bioanalytical method validation and study sample analysis-https://www.ema.europa.eu/en/documents/scientific-guideline/ich-guideline-m10-bioanalytical-method-validation-step-5_en.pdf

Chemicals which can be used for calibration curve preparation include Glutathione (e.g., Merck cat no. Y0000517, ThermoScientific™ cat no. 78259, Spectrum cat no. GL146 or the one available in the Promega kits discussed herein) and for ROS calibration curve—H₂O₂ (e.g., Sigma H1009, ThermoScientific).

In a further aspect, step (iv) of the method described herein comprises at least the following steps:—
(a) Add a control sample of medium only and an equal amount of compound interference controls to an assay plate;
(b) Add standard curve to at least 10 wells of the assay plate;
(c) Add a reagent which can reveal the signal from luciferin-luciferase systems to all wells;
(d) Shake assay plate for about 1 minute at room temperature at about 300 rpm;
(e) Incubate the assay plate for about 20 minutes at room temperature.
(f) Measure luminescence

Suitably step (f) is carried out using a luminescence plate reader.

Suitably the shaking in step (d) can last from about 10 seconds to about 5 minutes, suitably about 30 seconds to about two minutes at about 250-350 rpm, most suitably 300-310 rpm A further aspect of the invention comprises a kit comprising the GSH-Glo™ assay from Promega, plus a reducing agent capable of reduction of disulfide bonds such as dithiothreitol (DTT), dithioerythritol, dithionite, sodium borohydride, potassium borohydride, tris(2-carboxyethyl)phosphine (TCEP), triphenylphosphine or tributylphosphine, and instructions. Suitably the kit of the invention further comprises the ROS-Glo™ H₂O₂ assay from Promega. The methods described herein were tested using menadione to create oxidative stress in cells. Menadione (Vitamin K3) is a synthetic analogue of 1,4-naphthoquinone with a methyl group in the 2-position. Natural menadione (Vitamin K3) is fat-soluble while menadione sodium bisulfite is a synthetic form of menadione that is water-soluble. Menadione is used as a phosphatase inhibitor and an inhibitor of mitochondrial DNA polymerase y (pol y). Menadione can be used as an oxidative injury inducing agent.

The methods described herein are assays which combine and modify two commercially available kits from Promega.

The GSH/GSSG assay is based on GSH/GSSG-Glo™ assay kit catalogue #V6611 and V6612 and described in technical manual #TM344 from Promega. This manual is available here. https://web.archive.org/web/20230421070742/https:/ch.promega.com/-/media/files/resources/protocols/technical-manuals/101/gsh-gssg-glo-assay-protocol.pdf?rev=cfd4feb.992845f298e63d80cc9c4ed8&sc_lang=en

An alternative version of this method can be run using the GSH-Glo™ Glutathione assay kit catalogue #V6911 and V6912 and described in technical manual #TB369 from Promega. This manual is available here.

https://ch.promega.com/-/media/files/resources/protocols/technical-bulletins/101/gsh-glo-glutathione-assay-protocol.pdf?rev=2aa5c77ea6a840b39aeecd94ebb5473b&sc_lang=en

As noted above, a reducing agent was added to these methods and the sample split in two to enable calculation of the amount of GSH and GGSH from the same sample.

The ROS endpoint assay described herein is based on non-lytic variant of ROS-Glo™ H₂O₂ assay kit catalogue #G8820 and G8821 and described in technical manual TM391 from Promega. This manual is available here.

https://ch.promega.com/-/media/files/resources/protocols/technical-manuals/101/ros-glo-h2o2-assay-protocol.pdf?rev=f40c6e59ae684980b36441078f53a480&sc_lang=en Wherein “assay from Promega” is referred to here, it is intended to refer to one of these assays and/or a kit comprising the necessary regents, and/or apparatus, and/or instructions.

Combination of the ROS non-lytic (non-destructive) variant of the protocol allows the running of this assay with the GSH/GSSH assay on the same sample. It also allows for multiple timecourse characterization of ROS by collecting medium samples for each time point to be assessed. These multiple time course sampling methods have not been previously described. The invention is further described in the Examples below, which are provided to describe the invention in further detail. These examples, which set forth a preferred mode presently contemplated for carrying out the invention, are intended to illustrate and not to limit the invention.

### EXAMPLES

### Example 1

### Tissue Exposure and Endpoint Sample Collection

1. A tissues sample is treated basolaterally in medium containing 25 μM ROS-Glo™ H₂O₂ substrate for the desired exposure duration.
   Treatment duration is not longer than 6 hours due to instability of the substrate.
2. After treatment, 75 μL of the medium is collected for the ROS endpoint (see example 3), and the remaining cell or tissue sample transferred to new 24-well plate for the GSH/GSSG endpoint. ROS samples are stored at 4° C. for a maximum total of 6 hours taking into account the treatment duration, while GSH/GSSG samples must be processed immediately.

### Example 2

### GSH/GSSG Assay

This Specific Example is Based on GSH-Glo™ Assay Kit Catalogue #V6911 and V6912 and Described in Technical Bulletin #TB369 from Promega.
1. Starting with the cell or tissue sample from point 2 of Example 1, 100 μL GSH-Glo™ (1×) reagent is added to each tissue insert in a 24-well plate and shaken for 5 minutes at 300 rpm at room temperature.
2. 100 μL GSH-Glo™ (1×) reagent is added to standard curve wells in the assay plate.
3. 25 μL GSH-Glo™ (1×) reagent is added to Bond Breaker™ Tris (2-carboxyethyl) phosphine (TCEP) (Thermo Fisher Scientific, Massachusetts, USA) and to one or more water vehicle background control wells in the assay plate.
4. Each well of tissue lysate (100 μL) is split into two wells (25 μL each) in the assay plate, one for addition of TCEP and the other for water.
5. 2.5 μL of Bond Breaker™ TCEP is added to one of the pairs of lysate wells to measure total GSH, and the same is added to the TCEP background control well.
6. 2.5 μL H₂O is added to the lysate well to the other of the pair of lysate wells to measure reduced GSH, and the same was added to the one or more water vehicle background control well.
7. The well plate is shaken for 1 minute at 300 rpm at room temperature.
8. The well plate is incubated for 24 minutes standing at room temperature.
9. 25 μL of Luciferin Detection Reagent is added to all sample wells and to the TCEP and water vehicle background control wells and 100 μL of Luciferin Detection Reagent was added to standard curve wells.
10. The well plate is shaken for 1 minute at 300 rpm at room temperature.
11. The well plate is incubated for 14 minutes standing at room temperature.
12. The luminescence is measured, preferably with FLUOstar® Omega using the pre-defined “GSHG assay” protocol.

### Example 3

### GSH/GSSG Endpoint Analysis

1. The Relative Luminescence Units (RLU) are calculated by subtracting the appropriate background RLU to the measured raw RLU.
2. The GSSG RLU is calculated using the following formula: GSSG=(total glutathione RLU−reduced GSH RLU)/2., where total GSH is taken from the TCEP treated-samples and the reduced GSH is taken from the water-treated samples. During the reduction reaction 1 mole of GSSG produces 2 moles of GSH equivalents. Therefore, dividing the GSH signal by two gives the signal of GSSG.
3. The GSH/GSSG Ratio is determined using the calculated GSSG RLUs and the measured GSH RLUs.

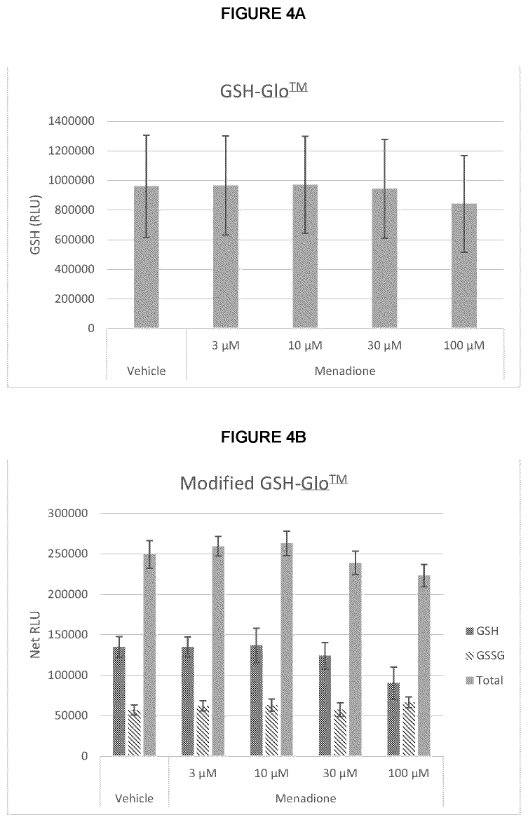

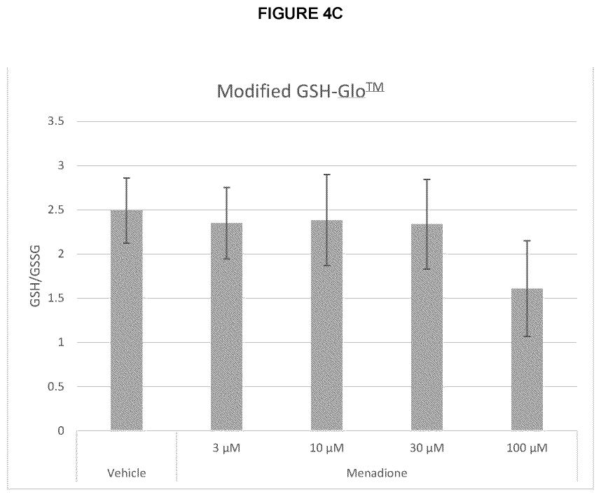

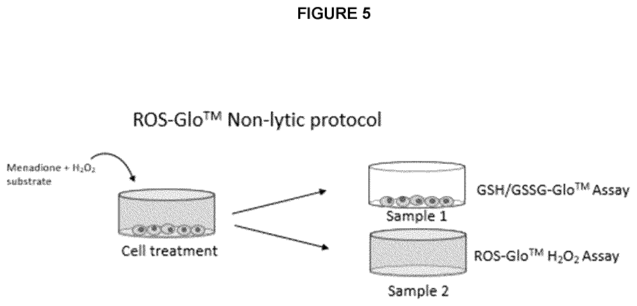

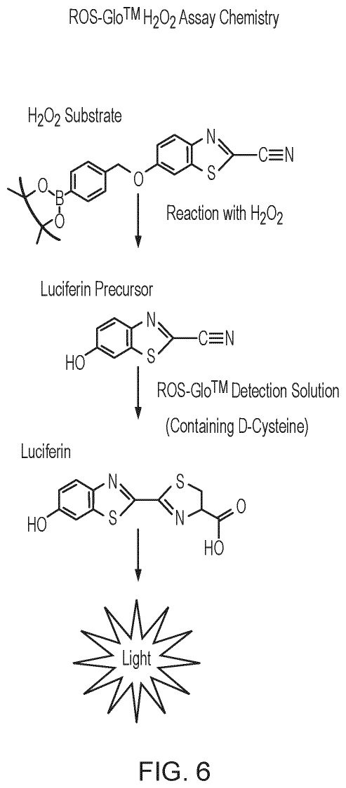

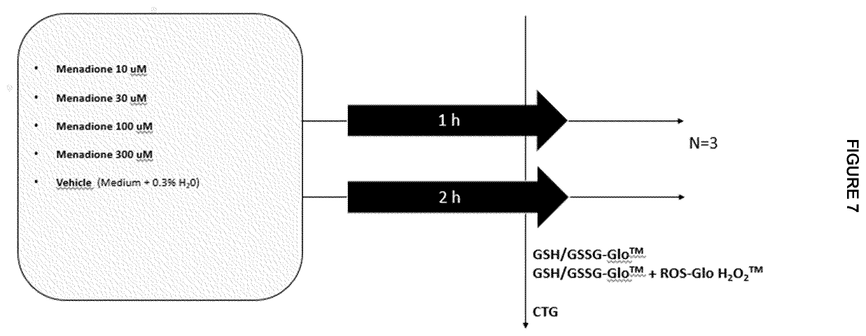

The results of examples 1-3 are shown in FIGS. 4B and 4C. FIG. 4A shows the results using the unaltered with GSH-Glo™ assay and kit for comparison.

Oxidative stress was caused by adding menadione to the cell or tissue sample before the assays were conducted.

Reference GSH/GSSG rations for different cell types are provided by Promega in Table 1 below.

TABLE 1

Ratio GSH/GSSG

| Cell Line | Cells/Well | Vehicle | Menadione |
|---|---|---|---|
| HeLa | 5,000 | 63.8 | 2.2 |
| HepG2 | 10,000 | 92.6 | 2.2 |
| HepaRG ® | 50,000 | 35.3 | 2.5 |
| A549 | 5,000 | 87.9 | 10.9 |
| Rat hepatocytes | 50,000 | 19.8 | 1.1 |

FIGS. 4A, 4B and 4C show there is significant variability in quantification of GSH levels with GSH-Glo™ Kit. Unexpectedly low GSH and high GSSG levels are detected with the modified (TCEP added) GSH-Glo™ Kit. The obtained GSH/GSSG ratio is very low for vehicle. Overall variability and low GSH compared to GSSG and total glutathione may arise from GSH auto-oxidation during sample processing.

Similar results were obtained with 3D bronchial epithelial cells (data not shown).

### Example 4

### GSH/GSSG Assay-Alternative Method

This Specific Example Based on GSH/GSSG-Glo™ Assay Kit Catalogue #V6611 and V6612 and Described in Technical Manual #TM344 from Promega
(A) Starting with the cell or tissue sample from point 2 of Example 1, the cell supernatant is removed.
(B) 100 μL of cold Passive lysis buffer is 1× diluted in water is added per well, to tissue/cell samples and shaken for 5 min at 300 rpm on ice.
(C) 100 μL of lysate is placed into 2 wells containing 50 μL each.
(D) In one well 1 μL of Luciferin-NT is added for quantification of total GSH.
(E) In the other well, 1 μL of Luciferin-NT and 0.5 μL of N-Ethylmaleimide (NEM) at 25 mM is added for quantification of oxidized GSSG.
(F) Both wells are incubated for 5 min shaking at 300 rpm at room temperature.
(G) 50 μL of Luciferin Generation Reagent is added per well, and both wells shaken for 1 min.
(H) Both wells are incubated for 29 min standing at room temperature.
(I) 100 μL of Luciferin detection reagent is added to both wells and they are shaken for 1 min.
(J) Both wells are incubated for 14 min.
(K) Both wells are transferred to an assay plate containing a calibration curve (suitably prepared as in the supplier's protocol).
(L) The luminescence of each well is measured with FLUOstar® Omega “GSHG assay”

This Example can be followed by the endpoint analysis of Example 5.

### Example 5

### ROS Endpoint

This Specific Example is Based on Non-Lytic Variant of ROS-Glo™ H₂O₂ Assay Kit Catalogue #G8820 and G8821 and Described in Technical Manual TM391 from Promega.
(a) 75 μL of sample medium collected in Example 1, step 2, and 75 μL of compound interference controls are added to an assay plate.
(b) 10 wells, 100 μL per well are added to the assay plate for standard curve calibration.
(c) 75 μL of freshly prepared ROS-Glo Detection Solution™ is added to all samples and compound interference control wells. D-cysteine is added on top if not present in reagents, preferably 0.01 to 20 mM, preferably 0.1 to 10 mM.
(d) 100 μL of ROS-Glo Detection Solution™ is added to standard curve wells.
(e) The assay plate is shaken for 1 minute at room temperature on a plate shaker set at 300 rpm.
(f) The plate is incubated for 20 minutes standing at room temperature.
(g) Luminescence is measured with FLUOstar® Omega using the pre-defined “ROS-Glo™ H₂O₂ assay”.

All shaking during the example may be carried out at +/−10 rpm to the amount stated. Room temperature was 18-25° C.

### Example 6

FIG. 7 is a schematic showing the experimental protocol used in Example 6.

In this example oxidative stress was simulated by adding menadione sodium bisulfite to four samples. Four of the samples had 10 μM, 30 μM, 100 μM or 300 μM of menadione sodium bisulfite respectively added to the medium to simulate increasing levels of oxidative stress. The fifth sample was used as the vehicle control wherein on the medium and 0.3% water were added. The assays were performed in triplicate on NHBE cells.

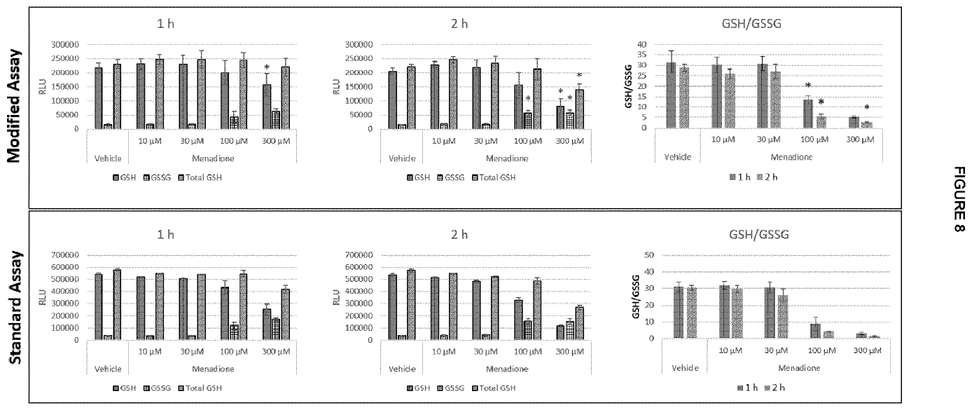

The results of Example 6 are shown in FIG. 8.

The top row shows the results of carrying out the assays of the invention after 1 hour of incubation with menadione sodium bisulfite (left-hand panel) and 2 hours of incubation with menadione sodium bisulfite (right-most panel). The right-hand panel shows the GSH/GSSG ration after 1 hour (dark bars) and 2 hours (light bars) for each sample. A star (*) indicates significance P<0.05 paired t-test.

In direct comparison, the bottom row of FIG. 8 shows the same cell cultures and times points as the top row, but in this case using the unmodified assays describe herein. As can be seen, the results are very similar. This indicates that the current invention has no detrimental effect on the results.

### Example 7

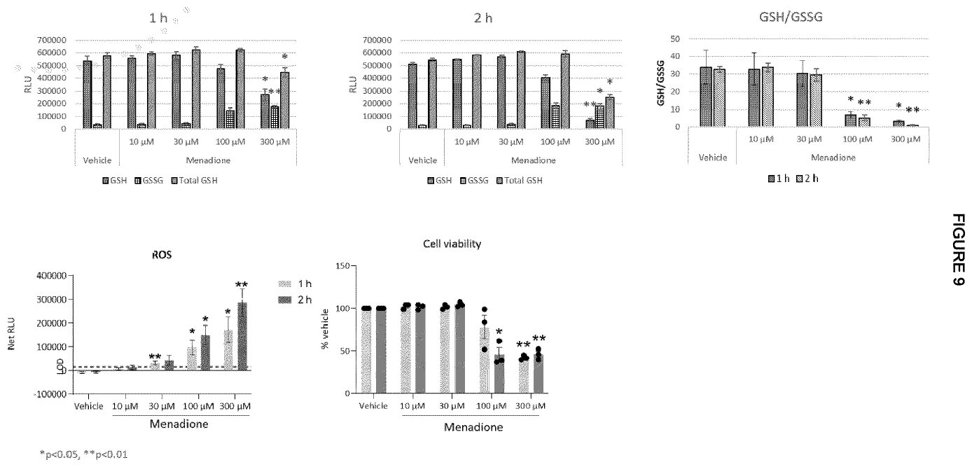

FIG. 9 shows a dosage effect on the experiments of Example 6 (as shown in FIG. 8).

In the top row of FIG. 9, the same experiments of the invention as per Example 6 (shown top row of FIG. 8) were carried out. In addition, the ROS endpoint analysis method of the invention (as per Example 5 above) was carried out on the same samples at the 1 hour and 2 hour time points (shown in FIG. 9, bottom row). A star (*) indicates significance P<0.05 paired t-test. Two stars (**) indicates significance P<0.01 paired t-test.

The cells used where an NHBE cell line exposed to water-soluble menadione sodium bisulfite.

As can be seen from the bottom row of FIG. 9, 30 UM Menadione sodium bisulfite increased ROS but caused no changes in GSH/GSSG. In other words, there was no change in oxidative stress levels. However, 100 μM Menadione increased ROS and was accompanied by decreased GSH/GSSG. This shows low intensity oxidative stress in the cells. The highest dose of Menadione (300 μM) induced highest ROS and lowest GSH/GSSG (6-fold reduction). This result is associated with decreased cell viability, and indicative of strong oxidative stress. Longer exposure to Menadione is associated with more oxidative stress and cytotoxicity (as determined by ATP levels), as shown by the comparison of the 1 hour and 2 hour time points.

### Example 8

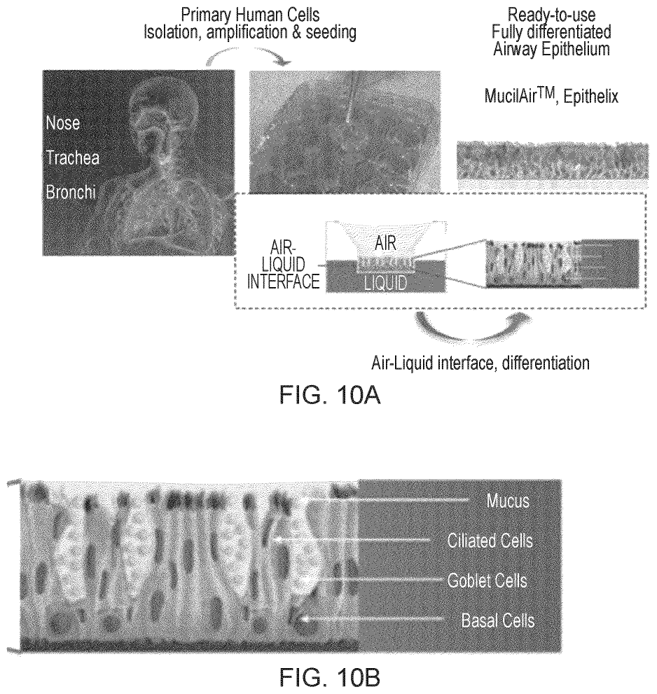

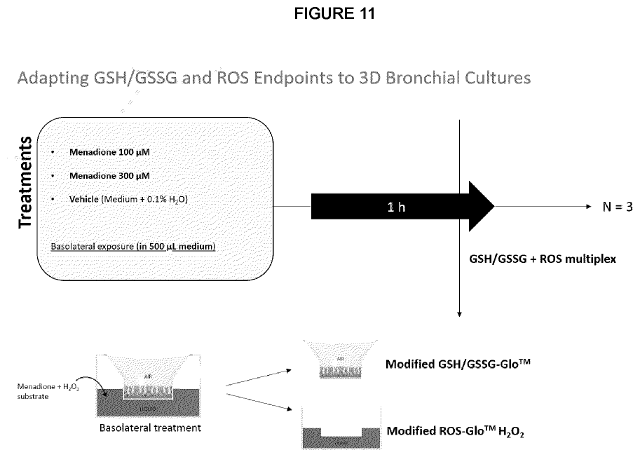

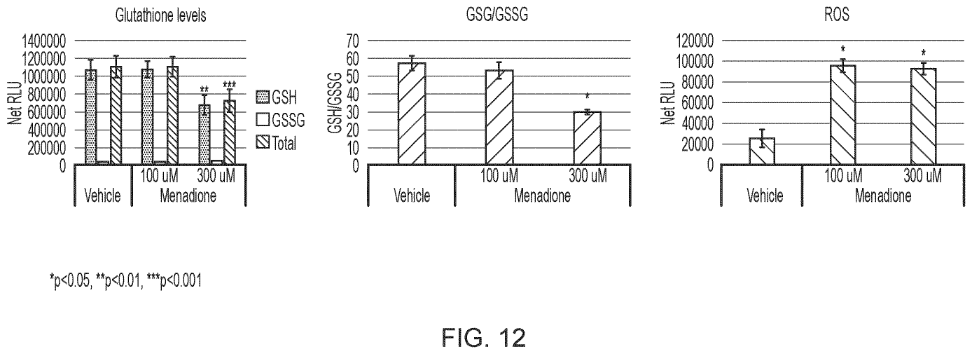

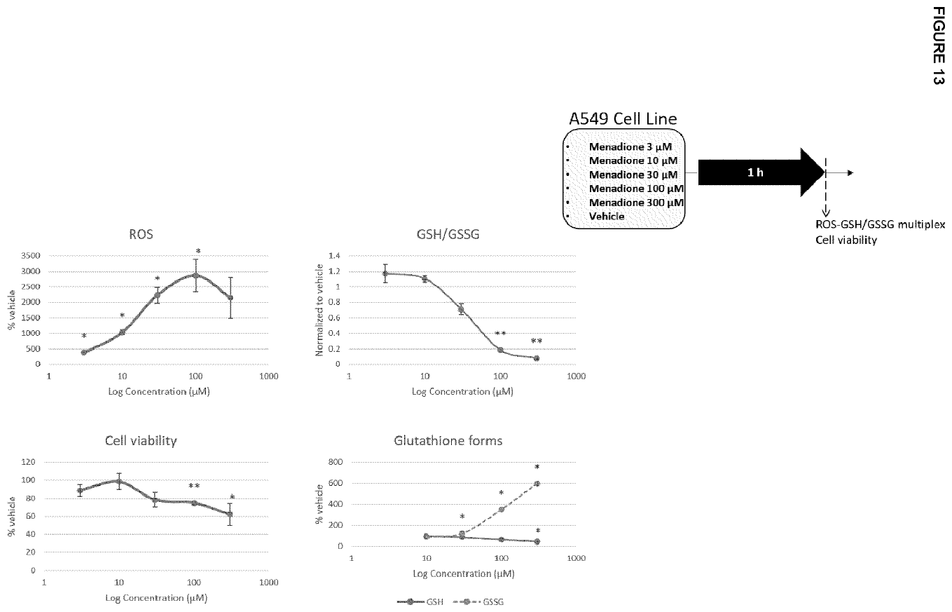

FIG. 10A provides details on the 3D bronchial culture test system used in FIG. 11 and FIG. 12, including the culturing conditions and the cell type constitution upon differentiation of primary cells. FIG. 10 B shows an enlarged section of FIG. 10A.

A 3D bronchial culture can be provided by isolating bronchial cells or using commercially available primary cells, culturing them and seeding them on a 3D framework. Suitable cultures include MucilAir™. MucilAir™ is an in vitro cell model of the human airway eplithelium cultures at the air liquid interface and available from Epithelix (for example, here https://www.epithelix.com/products/mucilair).

FIG. 11 is a further schematic showing how GSH/GSSG and ROS assays (as detailed in Example 4 and Example 5 above) can be adapted to 3D bronchial cultures. In this experiment, three 3D cultures were used. One had 100 μM of menadione sodium bisulfite added to simulate oxidative stress, the second had 300 μM menadione sodium bisulfite added and the third was a vehicle control wherein only the medium and water were added. The bottom of the figure shows how the samples to be assessed by the modified GSH/GSSG-Glo and ROS-Glo assays were collected from the 3D bronchial culture. The GSH/GSSG-Glo assays were performed on samples from the cell culture within the culture insert and the ROS-Glo assays were performed on the basolateral medium. This was performed in triplicate.

FIG. 12 shows the results of the experiment carried out in FIG. 11. (The significance levels are shown at the bottom of the figure). The GSH/GSSG ratio for the vehicle control for both the adapted GSH/GSSG and ROS assays as per the invention is reproducible and similar to that seen in 2D conditions, indicating that the protocol is also working for 3D cell culture. Lower menadione sodium bisulfite dose significantly increased ROS but had no effect on GSH/GSSG. Higher dose further decreased GSH and GSH/GSSG but had no further impact on ROS and GSSG.

### Example 9

FIG. 13 shows the results from carrying out the assays of the invention as per Example 4 and Example 5 to test their applicability to a different cell line.

In this experiment, an A549 lung cell line was exposed to lipid-soluble menadione (Vitamin K3) to simulate oxidative stress. This induced a similar result to the NHBE cell line exposed with water-soluble menadione sodium bisulfite from FIG. 9, i.e. increase in ROS and decrease in GSH/GSSG in a dose-dependent manner, with effects on cell viability at the two highest concentrations.

A star (*) indicates significance P<0.05 paired t-test. Two stars (**) indicates significance P<0.01 paired t-test.

### Example 10

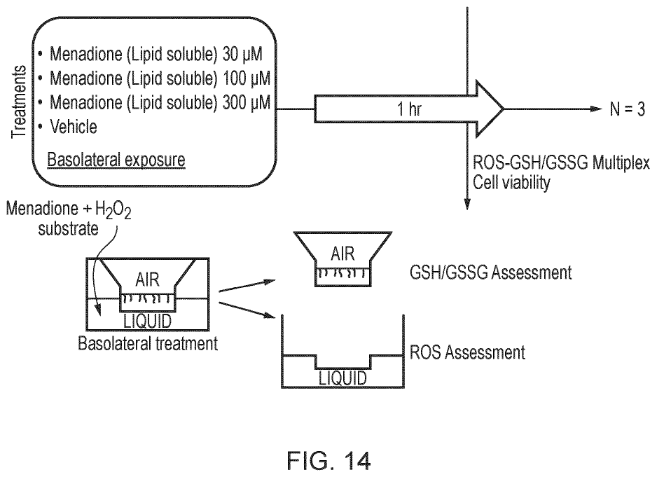

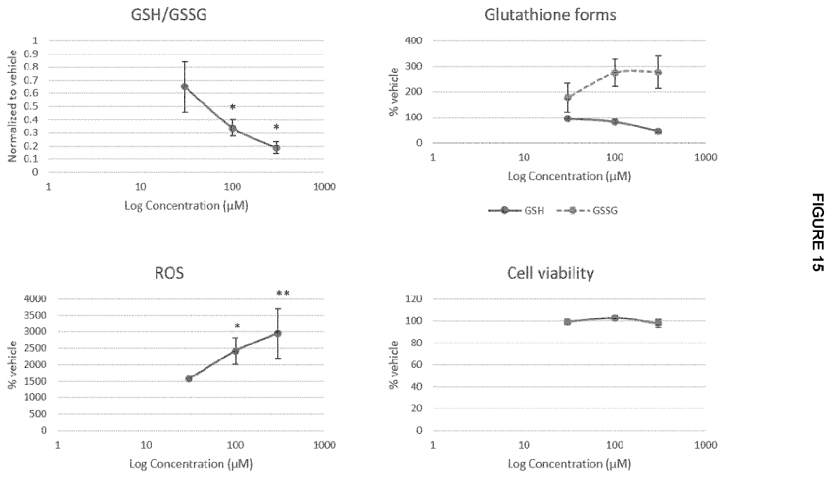

FIG. 14 is a schematic showing the experimental protocol which obtained the results of FIG. 15 in 3D bronchial cultures by carrying out the assays of the invention as in Example 4 and Example 5 using lipid-soluble menadione.

A 3D bronchial culture as per Example 8 was used.

FIG. 15 shows dose-dependent decrease in GSH and GSH/GSSG, and increase in GSSG and ROS upon exposure of 3D bronchial cultures to lipid-soluble menadione, with no effects on cell viability, which is in contrast to the results from FIG. 11 using menadione sodium bisulfite. This indicates that the current invention could capture distinct effects on glutathione levels induced by different chemical forms of menadione.

### Overall Results.

The disclosed method addresses known issues with the modified kits described.
This approach saves time for tissue treatment and cost (1 tissue per condition instead of 3=66% cost reduction).
Cost reduction is especially significant for 3D cell culture work, with a dropoff around two thirds in cost.
The methods of the invention showed time-dependent effects.
This holistic approach has increased biological relevance as the 3 oxidative stress readouts originate from the same sample, eliminating biological (tissue-to-tissue) variability, while also minimizing technical variability.
It provides a scientifically comprehensive characterization of oxidative stress, which per definition, is not possible by only assessing a single biomarker.
The methods disclosed are compatible with high-throughput assessment and less prone to compound interference.

Any publication cited or described herein provides relevant information disclosed prior to the filing date of the present application. Statements herein are not to be construed as an admission that the inventors are not entitled to antedate such disclosures. All publications mentioned in the above specification are herein incorporated by reference. Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in engineering, cellular biology and molecular biology or related fields are intended to be within the scope of the following claims.

1.-15. (canceled)

16. A method of measuring oxidative stress in a cell or tissue sample comprising the steps of:
(i) treating the cell or tissue sample in a medium;
(ii) collecting a portion of the medium;
(iii) measuring amounts of glutathione in the cell or tissue sample; and
(iv) measuring amounts of reactive oxygen species (ROS) in the media portion collected in step (ii),
wherein step (iii) is carried out immediately after steps (i) and (ii), and
wherein step (iv) is carried out simultaneously with step (iii) or within 6 hours of step (iii) including assay time if the collected media portion is stored at about 0-8° C. until step (iv) is carried out.

17. The method according to claim 16, wherein the ROS amount and/or glutathione amount is measured using luminescence.

18. The method according to claim 16, wherein the medium of step (i) comprises a hydrogen peroxide (H₂O₂) substrate.

19. The method according to claim 16, wherein a treatment duration in step (i) is about 6 hours or less.

20. The method according to claim 16, wherein the amount of glutathione measured in step (iii) is a sum total of reduced glutathione (GSH), and oxidised GSH (GSSG).

21. The method according to claim 16, wherein a GSH/GSSG ratio is calculated in step (iii) using relative luminescence units (RLU).

22. The method according to claim 16, wherein a reducing agent configured to reduce disulfide bonds is used to measure GSH amounts in step (iii).

23. The method according to claim 16, wherein step (iii) comprises at least the following steps:
(a) placing the cell or tissue sample in a well plate, adding a reagent comprising a luciferin-NT substrate and a glutathione S-transferase (GST) enzyme (a GSH-Glo™ reagent) to each well, and shaking for about 5 minutes at about 300 rpm at room temperature to lyse the cells;
(b) adding the GSH-Glo™ reagent to standard curve wells in the assay plate;
(c) adding the GSH-Glo™ reagent to a reducing agent and one or more the water vehicle background control wells in the assay plate, and adding D-cysteine if not present in the reagents;
(d) splitting each well containing the lysed cell or tissue sample into two wells in the assay plate;
(e) to measure total glutathione, adding the reducing agent to one of each of the pairs of sample wells and to at least one reducing agent background control well;
(f) to measure reduced GSH, adding water to the remaining sample wells that do not contain the reducing agent, and to the one or more water vehicle background control wells;
(g) shaking all wells for about 1 minute at about 300 rpm at room temperature;
(h) incubating all wells at room temperature;
(i) adding a reagent that can reveal a signal from luciferin-luciferase systems to the sample wells, to the at least one reducing agent well, to the one or more water vehicle background control wells, and to the standard curve wells;
(j) shaking all wells for about 1 minute at about 300 rpm at room temperature;
(k) incubating all wells at room temperature; and
(l) measuring luminescence.

24. The method according to claim 23, wherein the reducing agent is configured to reduce disulphide bonds.

25. The method according to claim 24, wherein the reducing agent is dithiothreitol (DTT), dithioerythritol, dithionite, sodium borohydride, potassium borohydride, tris(2-carboxyethyl)phosphine (TCEP), triphenylphosphine, or tributylphosphine.

26. The method according to claim 23, wherein step (1) is carried out using a luminescence plate reader.

27. The method according to claim 16, wherein step (iii) comprises at least the following steps:
(a) removing cell supernatant from the sample;
(b) adding passive lysis buffer to the sample and shaking for about 5 min at about 300 rpm;
(c) dividing the sample equally between two wells;
(d) in one well adding Luciferin-NT for quantification of total glutathione;
(e) adding Luciferin-NT and N-Ethylmaleimide (NEM) to the other well for quantification of oxidized glutathione (GSSG);
(f) incubating both wells for about 5 min shaking at about 300 rpm at room temperature;
(g) adding Luciferin Generation Reagent to each well and shaking for about 1 min;
(h) incubating both wells at room temperature;
(i) adding a reagent that can reveal the signal from luciferin-luciferase systems to both wells and shaking for about 1 min;
(j) incubating both wells for about 14 min;
(k) transferring sample wells to an assay plate containing a calibration curve; and
(l) measuring luminescence.

28. The method according to claim 27, wherein step (1) is carried out using a luminescence plate reader.

29. The method according to claim 16, wherein step (iv) comprises at least the following steps:
(h) adding a control sample of medium only and an equal amount of compound interference controls to an assay plate;
(i) adding standard curve to at least about 10 wells of the assay plate;
(j) adding a reagent that can reveal a signal from luciferin-luciferase systems to all wells, add D-cysteine if not already present in the reagent;
(k) sharking the assay plate for about 1 minute at room temperature at about 300 rpm;
(l) incubating the assay plate for about 20 minutes at about room temperature; and
(m) measuring luminescence.

30. A kit comprising a luminescent assay to detect and measure reduced glutathione (GSH) in biological samples like cells and tissues (GSH-Glo™ assay kit or GSH/GSSG-Glo™ kit from Promega), a reducing reagent, and instructions.

31. The kit according to claim 30, further comprising a bioluminescent assay that measures hydrogen peroxide (H₂O₂), a type of reactive oxygen species (ROS), in cell cultures or enzyme reactions (the ROS-Glo™ H₂O₂ assay from Promega).
